(19) United States
(12) Patent Application Publication
(10) Pub. No.: US 2012/0271107 A1
(43) Pub. Date: Oct. 25, 2012

(54) SELF-PROPELLED DEVICE FOR ENDOSCOPE

(75) Inventors: Shinichi YAMAKAWA, Kanagawa (JP); Tsuyoshi ASHIDA, Kanagawa (JP); Takayuki NAKAMURA, Kanagawa (JP); Yasunori OHTA, Kanagawa (JP); Rick CORNELIUS, Wayzata, MN (US); John J. ALLEN, Mendota Heights, MN (US)

(73) Assignee: FUJIFILM CORPORATION, Tokyo (JP)

(21) Appl. No.: 13/454,796

(22) Filed: Apr. 24, 2012

Related U.S. Application Data
(60) Provisional application No. 61/478,840, filed on Apr. 25, 2011.

(51) Int. Cl. A61B 1/00

(52) U.S. Cl. 600/114

(57) ABSTRACT

A self-propelled device includes a mounting part mounted on an insertion part of an endoscope, a hollow toroidal rotary body, and a rotary body supporting tube that is arranged in the internal space of rotary body and supports the rotary body from the inside. The rotary body supporting tube is attached to the outside of the mounting part. The mounting part is provided with a worm wheel, and the rotary body supporting tube is provided with a driven roller. The rotary body is sandwiched between the worm wheel and the driven roller, and is circulated and moved with the rotation of the worm wheel. Wipers for closing the gap formed between the mounting part and the rotary body are provided at the front and rear ends of the mounting part, and thereby preventing entering of foreign body from the gap.

### BACKGROUND OF THE INVENTION

1. Field of the Invention

The present invention relates to a self-propelled device for an endoscope that assists in insertion of the endoscope into a body cavity.

2. Description of Related Art

Endoscopes are widely used for observation or medical treatment in body cavities. This kind of endoscope includes an insertion part to be inserted into a body cavity, and a manipulating part that manipulates this insertion part, and the insertion part is inserted into the body cavity by manipulating the manipulating part. In the endoscope, the insertion part is inserted into the body cavity while manipulating the manipulating part to curve the distal end portion of the insertion part, however extensive experience is required for insertion thereof. For example, the procedure of inserting the insertion part in a part that is not fixed to the body cavity like a sigmoid colon or a transverse colon is difficult, and when the insertion skill is inexperienced, the patient will undergo significant pain. For this reason, a self-propelled device for an endoscope that propels the endoscope in the insertion direction within the intestinal tract is proposed as described in Japanese Patent Translation Publication No. 2009-513250. In this apparatus, a hollow toroidal rotary body is attached to the distal end of the insertion part of an endoscope, and this rotary body is circulated in the longitudinal direction of the insertion part to draw the insertion part into the depths of the intestinal tract. The rotary body abuts on a driving roller arranged between the outer periphery of the insertion part, and the rotary body, and is circulated with the rotation of the driving roller.

However, in the apparatus described in Japanese Patent Translation Publication No. 2009-513250, there is a concern that foreign body (for example, digested materials, the inner wall of the intestinal tract, or the like) may be drawn-in between the outer periphery of the insertion part and the rotary body, with the circulation of the rotary body.

### SUMMARY OF THE INVENTION

An object of the invention is to provide a self-propelled device for an endoscope that can prevent drawing-in of foreign body.

In order to achieve the above object, the self-propelled device for an endoscope of the invention includes a mounting part, a rotary body, a supporting part, a driving roller, and first and second wipers. The mounting part is detachably mounted on the insertion part of the endoscope. The rotary body is formed in a hollow toroidal shape or obtained by forming a belt in a ring shape. The supporting part has at least a portion arranged in an internal space of the rotary body and supports the rotary body so as to be able to circulate along the longitudinal direction of the insertion part. Additionally, the supporting part is formed in the shape of a tube that surrounds the mounting part and is attached to the outside of the mounting part with the rotary body supported. The driving roller is provided at the mounting part so as to come into contact with the rotary body. The driving roller circulates and moves the rotary body. The first and second wipers respectively arranged at the front and rear ends of the mounting part so as to close the gap between the mounting part and the rotary body. The first and second wipers slide on the rotary body when the rotary body rotates, thereby preventing drawing-in of foreign body between the mounting part and the rotary body.

The first and second wipers may be formed from a material having elasticity, and may be arranged in a state where the wipers are pressed against and elastically deformed by the rotary body. The first and second wipers are preferably arranged inside a turning point of the rotary body in a direction which the rotary body circulates and moves. The first wiper may be arranged with its surface facing the front end of the mounting part, and the angle between the surface and the rotary body may be formed to be equal to or more than 90°. Additionally, the second wiper may be arranged with its surface facing the rear end of the mounting part, and the angle between the surface and the rotary body may be formed to be equal to or more than 90°.

The wipers may be formed in a tapered shape whose thickness becomes smaller toward its tip where the wiper slides on the rotary body.

The first and second wipers may be formed in a ring shape, and may be attached to the mounting part at a position where the tip of each of the first and second wipers extends outside in a radial direction of the mounting part. The first and second wipers may have cross-sections along the central axis of the insertion part formed in a substantially L-shape or T-shape. When formed in T-shape, the first and second wipers have a triangular head where its broad surface comes in contact with the rotary body.

The first and second wipers may be formed from a biocompatible plastic. The first and second wipers may be detachably provided at the mounting part so as to be replaceable when the wipers have deteriorated through use. The mounting part may have an opening portion through which the insertion part is inserted, and may be mounted on the outer periphery of the insertion part as the insertion part is inserted through the opening portion.

The rotary bodies are endless belts obtained by forming a belt in a ring shape, and are juxtaposed around the longitudinal direction of the insertion part. When the rotary bodies are endless belts, preferably, the self-propelled device for an endoscope further includes a front end sealing part and a rear end sealing part. The front end sealing part prevents entering of foreign body from a front gap formed between the supporting part and the mounting part between a pair of the adjacent endless belts. The rear end sealing part prevents entering of foreign body from a rear gap formed between the supporting part and the mounting part between the pair of the adjacent endless belts. The self-propelled device for an endoscope further includes a side sealing part. The side sealing part prevents entering of foreign body from side gaps formed at both sides of the endless belt.

Since the self-propelled device for an endoscope of the invention provides the wipers that close the gap between the mounting part and the rotary body, it is possible to prevent drawing-in of the foreign body therebetween.

### BRIEF DESCRIPTION OF THE DRAWINGS

The above objectives and advantages will be able to be easily understood by those skilled in the art by reading the detailed description of a preferable embodiment of the invention with reference to the accompanying drawings:

FIG. 1 is a schematic view of an endoscope system;

FIG. 2 is a perspective view of a distal end portion of an endoscope and a body of a self-propelled device;

FIG. 3 is an exploded view of the body of the self-propelled device;

FIG. 4 is an exploded view of a mounting part;

FIG. 5 is a cross-sectional view showing a state where the body is cut in a plane perpendicular to a central axis CL of an insertion part;

FIG. 6 is a cross-sectional view taken along a line VI-VI of FIG. 5;

FIG. 7 is a partial cross-sectional view of a wiper having a cross-section of L-shape;

FIG. 8 is a partial cross-sectional view of a wiper having a cross-section of T-shape; and

FIG. 9 is a perspective view showing an example in which an endless belt is used as a rotary body.

### DETAILED DESCRIPTION OF PREFERABLE EMBODIMENTS

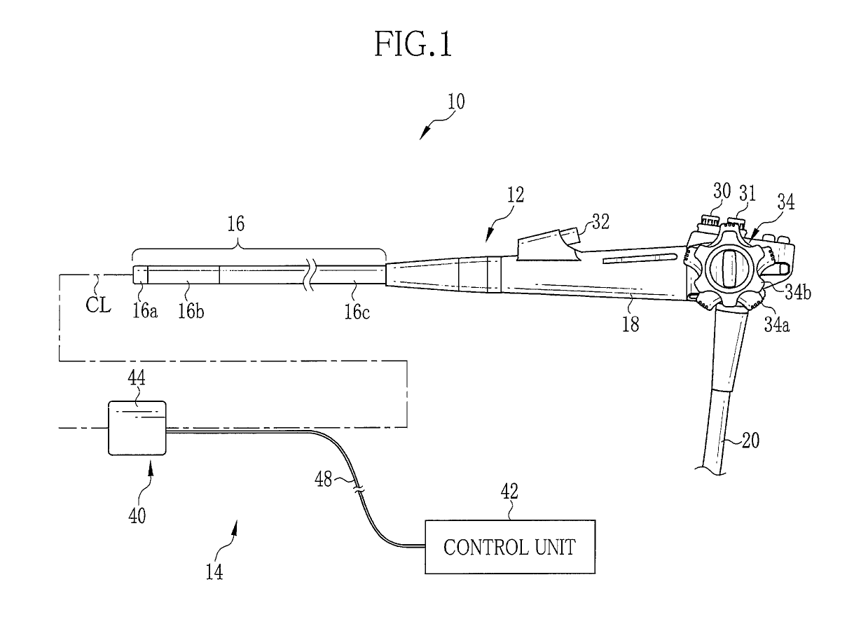

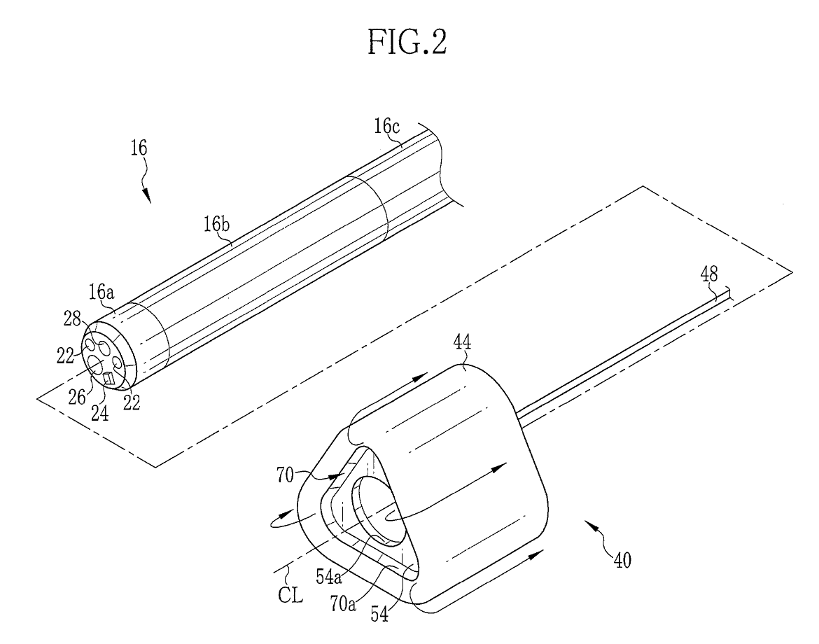

In FIGS. 1 and 2, an endoscope system 10 is constituted by an electronic endoscope 12, and a self-propelled device (endoscope insertion assisting device) 14 mounted on the electronic endoscope 12. The electronic endoscope 12 is constituted by an insertion part 16 to be inserted into a body cavity (for example, the large intestine), a manipulating part 18 continuously provided at the rear end of the insertion part 16, and a processor device, alight source device, an air and water supply device, or the like (none shown) connected to the manipulating part 18 via a universal cord 20.

The insertion part 16 includes a distal end rigid portion 16a, a curvable portion 16b, and a flexible tube portion 16c that are provided sequentially from the distal end (front) side. The distal end rigid portion 16a is provided with a pair of illumination windows 22 for allowing illumination light from the light source device to be radiated to a part to be observed therethrough, air supply and water supply nozzle 24 for jetting air and water to be supplied from air and water supply devices toward an observation window, and a forceps outlet 26 through which the distal end of a treatment tool, such as an electric scalpel inserted through a forceps port 32 to be described below, is exposed.

Additionally, the distal end rigid portion 16a is provided with an observation window 28 for taking in an image of a part to be observed within the body. An objective optical system, and a solid-state image sensing device, such as a CCD or a CMOS image sensor, are provided behind the observation window 28. The solid-state image sensing device is connected to the processor device (not shown) by a signal cable inserted through the insertion part 16, the manipulating part 18, and the universal cord 20. The processor device drives and controls the solid-state image sensing device to capture the image of a part to be observed, and displays the obtained image on a monitor (not shown).

The curvable portion 16b is capable of being curved, and is vertically and horizontally curved according to the manipulation of the manipulating part 18. This enables the distal end rigid portion 16a to be turned to a desired direction. The flexible tube portion 16c is deformable by a helical coil or the like, and is formed with a length of about several meters in order to allow the distal end rigid portion 16a to reach a target part within the body cavity.

The manipulating part 18 is provided with air supply and water supply buttons 30 and 31 for jetting air and water off from the aforementioned air supply and water supply nozzle 24, and the forceps port 32 through which a treatment tool, such as an electric scalpel, is inserted. Additionally, the manipulating part 18 is provided with an angle knob 34. The angle knob 34 is arranged such that two manipulation dials 34a and 34b are superimposed on each other. The curvable portion 16b can be curved up and down through a wire by rotating the manipulation dial 34a on the deep side, and the curvable portion 16b can be curved to the right and left through the wire by rotating the manipulation dial 34b on the near side.

The self-propelled device 14 is mounted on the electronic endoscope 12 and assists in the advance/retreat of the insertion part 16 of the electronic endoscope 12 within the body cavity. The self-propelled device 14 includes a body 40 that attached to the distal end side of the insertion part 16 and inserted into the body cavity, and a control unit 42 that is arranged out of the body cavity and drives and controls the body 40.

The body 40 includes a rotary body 44 formed in a hollow toroidal shape. The rotary body 44 is formed from a biocompatible plastic (polyvinyl chloride, polyamide resin, fluororesin, polyurethane resin, and the like) having flexibility. The rotary body 44 is supported so as to be able to circulate by a rotary body supporting tube 52 to be described below, and is circulated in a direction (longitudinal direction of the insertion part 16) parallel to the central axis CL. A propulsive force is given to the insertion part 16 as the rotary body 44 is circulated. In addition, an arrow of FIG. 2 indicates the circulation direction of the rotary body 44 when the insertion part 16 is moved to the front.

A torque wire 48 for supplying a driving force to the rotary body 44 and a tube (not shown) that covers the torque wire 48 are provided at the rear end of the body 40. The torque wire 48 and the tube have distal ends connected to the body 40 and rear ends connected to the control unit 42.

The control unit 42 is provided with a motor (not shown) that rotates the torque wire 48, and a manipulating section (not shown) for controlling the rotational direction or rotational speed of the motor, and the rotation of the rotary body 44 can be controlled, that is, the propulsive direction and propulsive speed of the insertion part 16 can be controlled by manipulating the manipulating part.

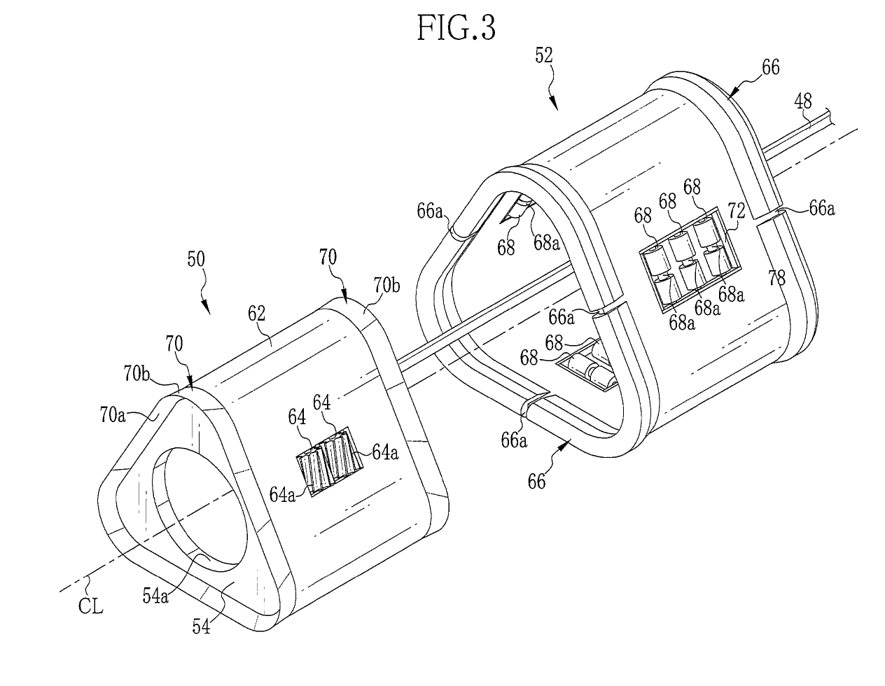

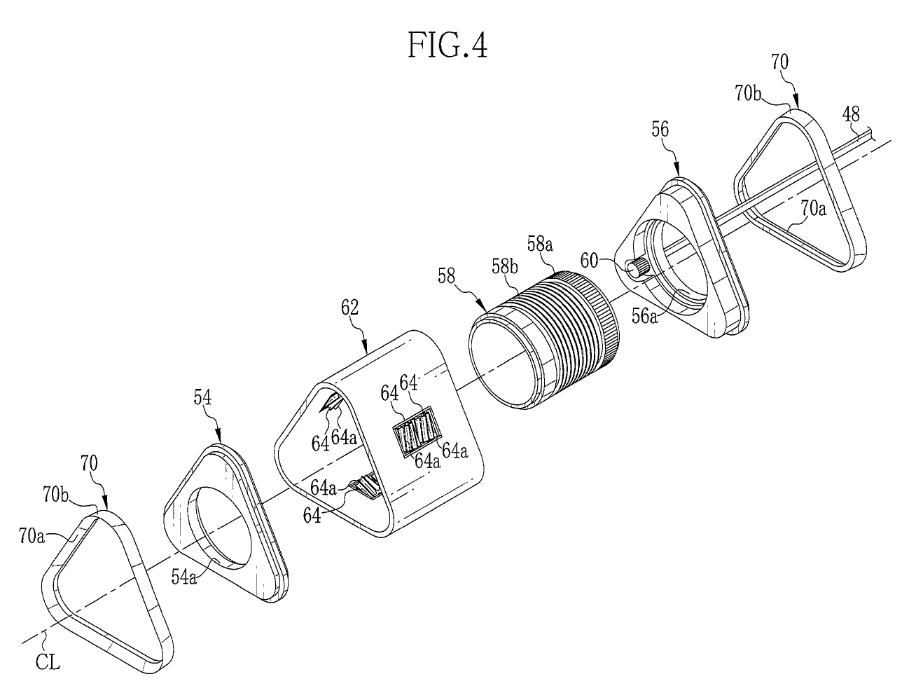

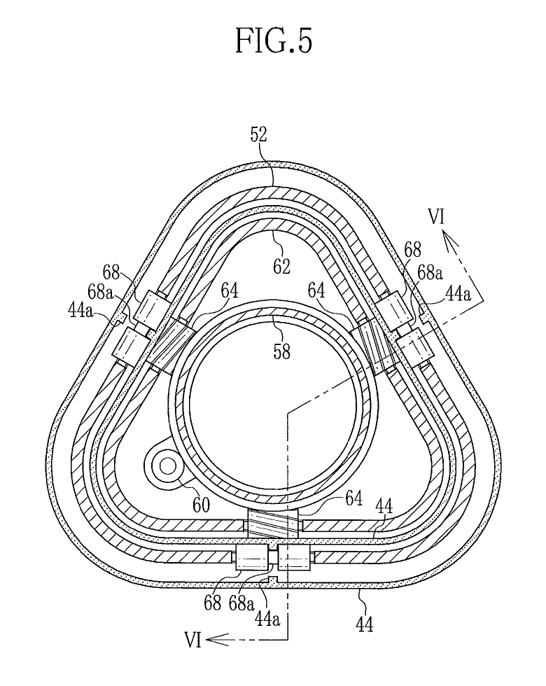

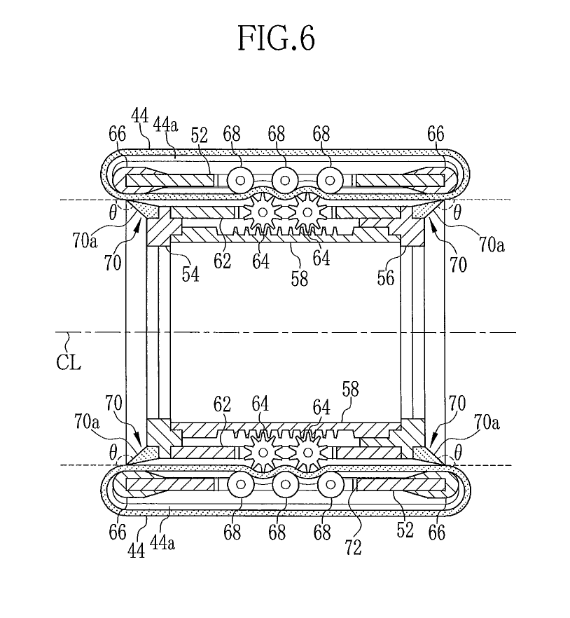

The configuration of the body 40 will be described below in detail with reference to FIGS. 3 to 6. In addition, the rotary body 44 is omitted in FIGS. 3 and 4. As shown in FIG. 3, the body 40 includes a mounting part 50 detachably provided at the insertion part 16, and a rotary body supporting tube (supporting part) 52 that supports the rotary body 44 and is mounted and supported outside the mounting part 50.

As shown in FIG. 4, the mounting part 50 includes a front plate 54 and a rear plate 56 respectively fixed to the front and rear ends of a wheel supporting tube 62. The front plate 54 and the rear plate 56 are formed with openings 54a and 56a through which the insertion part 16 is inserted, and the mounting part 50 is mounted on the outer periphery of the insertion part 16 so as not to fall out by fitting the insertion part 16 into the openings 54a and 56a.

A gear barrel (driving barrel) 58 is arranged between the front plate 54 and the rear plate 56. The gear barrel 58 is formed in the shape of a cylinder that surrounds the insertion part 16, and is rotatably supported around the central axis CL between the front plate 54 and the rear plate 56. A pinion gear 60 attached to the distal end of the torque wire 48 is rotatably supported by the rear plate 56, and the pinion gear 60 meshes with a spur gear 58a formed at the outer periphery of a rear end portion of the gear barrel 58. The gear barrel 58 rotates with the rotation of the pinion gear 60.

The wheel supporting tube 62 is formed in the shape of a substantially triangular tube. The wheel supporting tube 62 has an inner peripheral front end portion fitted to the outer peripheral rear end portion of the front plate 54 and an inner peripheral rear end portion fitted to the outer peripheral front end portion of the rear plate 56, respectively, and is united with these plates. Three square through holes are formed at intervals of 120 degrees around the central axis CL in the flat side walls of the wheel supporting tube 62, and a pair of front and rear worm wheels 64 is arranged at each through hole.

The worm wheels 64 are formed in a substantially columnar shape, and are rotatably supported around an axis perpendicular to the central axis CL. The outer periphery of each worm wheel 64 is formed with a tooth row 64a that meshes with worms 58b formed in the outer periphery of the gear barrel 58. The worm wheels 64 rotate with the rotation of the gear barrel 58.

As shown in FIGS. 3, 5, and 6, the rotary body supporting tube 52 is arranged outside the wheel supporting tube 62. The rotary body supporting tube 52 is formed in the shape of a triangular tube longer and having a larger diameter than the wheel supporting tube 62. Additionally, the rotary body supporting tube 52 is arranged in the internal space of the rotary body 44, and supports the rotary body 44 from the inside.

Bumpers 66 are provided at the front and end portions of the rotary body supporting tube 52, and the rotary body 44 directionally turns along the bumpers 66. The bumpers 66 are made from materials with a small frictional resistance with the rotary body 44 so that the rotary body 44 can smoothly turn at its turning point.

Additionally, each bumper 66 is formed with groove portions 66a, and linear projections 44a formed on the inner surface of the rotary body 44 are penetrated into the groove portions 66a. The linear projections 44a are continuously formed over the entire inner circumference of the rotary body 44 so as to run along the circulation direction of the rotary body 44. By slidably engaging the groove portions 66a with the linear projections 44a in this way, a problem that the rotary body 44 may rotate around the central axis CL of the insertion part 16 is prevented.

Through holes 72 passing through the flat side walls of the rotary body supporting tube 52 are provided between the bumpers 66 of the front and rear end portions of the rotary body supporting tube 52, and a set of three, that is, front, middle, and rear driven rollers 68 are arranged at each through hole 72. The driven rollers 68 are rotatably supported around the axis perpendicular to the central axis CL. Each driven roller 68 is formed with a groove portion 68a to which the linear projection 44a is fitted into, and the rotary body 44 running deviation is more reliably prevented by the close fit between the groove portions 68a and the linear projections 44a.

If the rotary body supporting tube 52 is attached to the outside of the wheel supporting tube 62, the driven rollers 68 are arranged alternately in front of and behind the worm wheels 64. This regulates forward and rearward movement of the rotary body supporting tube 52. Additionally, if the rotary body supporting tube 52 is attached to the wheel supporting tube 62, the rotary body 44 is sandwiched between the worm wheels 64 and the driven rollers 68 as well as being pressed against the worm wheels 64 by the driven rollers 68. Then, the rotary body 44 circulates and moves with the rotation of the worm wheels 64. Since each of the worm wheels 64 has relatively low teeth so as not to damage the rotary body 44, the worm wheels 64 work as the driving rollers with ribs.

Although a propulsive force is given to the insertion part 16 as the rotary body 44 circulates, if foreign body, such as the inner wall of the intestinal tract, is drawn-in between the rotary body 44 and the wheel supporting tube 62 with the circulation of the rotary body 44, not only the circulation of the rotary body 44 will be hindered, but also the burden on a patient will increase. Additionally, if foreign body of digested materials are adhered to the rotary body 44, the foreign body may get caught by the worm wheels (driving rollers) 64, which inhibits the smooth rotation of the driving rollers 64. In view of this, in the self-propelled device 14, a wiper 70 is provided at the mounting part 50, and a gap between the mounting part 50 and the rotary body 44 is closed by the wiper 70, and thereby preventing drawing-in of foreign body.

In FIGS. 3, 4, and 6, the wiper 70 is formed from a biocompatible plastic (polyvinyl chloride, polyamide resin, fluororesin, polyurethane resin, and the like) having elasticity. The wiper 70 is formed in a ring shape, and is attached to the outer peripheral distal end of the front plate 54 and the outer peripheral rear end of the rear plate 56 such that the wiper 70 extends outside in a radial direction of the front plate 54 and the rear plate 56.

The wiper 70 is formed with a tapered face 70a at its inner periphery to be formed in a conical shape. A tapered face 70b of the outer periphery has smaller inclination of the insertion part with respect to the central axis CL than the tapered face 70a of the inner periphery. Thereby, the wiper 70 is formed in such a tapered shape that the external diameter becomes larger and the thickness becomes smaller toward its tip. The wiper 70 is attached to the front plate 54 with the tapered face 70a turned to the front, and the wiper 70 is attached to the rear plate 56 with the tapered face 70a is turned to the rear.

The wiper 70 presses the rotary body 44 outward by the outer periphery, and closes the gap between the mounting part 50 and the rotary body 44 in a state where the wiper is elastically deformed by the pressing. When the rotary body 44 rotates, the wiper slides on the rotary body 44 to prevent foreign body from being drawn-in between the rotary body 44 and the mounting part 50. Additionally, the wiper 70 strips off foreign body adhered to the rotary body 44. In the present embodiment, since the angle θ formed between the tapered face 70a of the wiper 70 and the rotary body 44 is 90° or more, drawing-in of foreign body can be more reliably prevented as compared to a case where the angle formed between the tapered face 70a of the wiper 70 and the rotary body 44 is less than 90°.

In addition, in the invention, it is sufficient if the gap between the mounting part 50 and the rotary body 44 is closed by the wiper 70 to prevent drawing-in of foreign body therebetween. Thus, the detailed configuration is not limited to the above embodiment, and can be appropriately changed. For example, the wiper may be detachably to the mounting part so as to be replaceable when the wiper 70 has deteriorated.

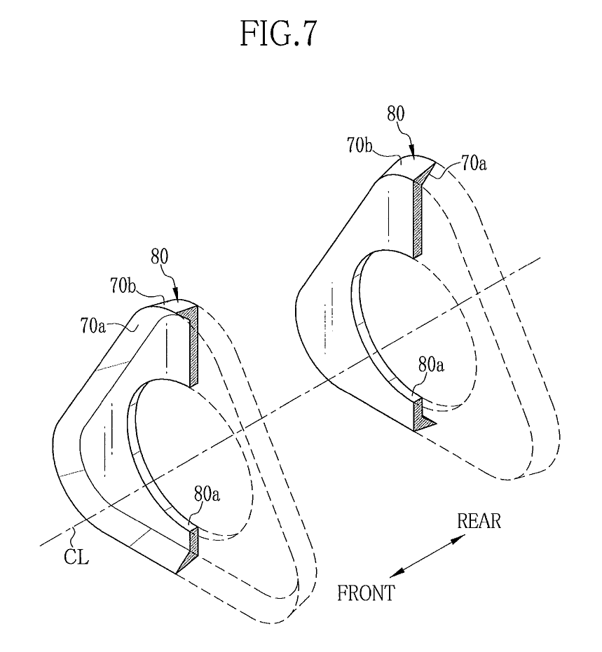

Additionally, although a substantially triangular ring-like wiper is used in the above embodiment, the shape of the wiper is not limited thereto. For example, as shown in FIG. 7, a wiper 80 whose cross-section is formed in a substantially L-shape may be provided to close the gap between the outer periphery of the insertion part 16, and the rotary body 44. The wiper 80 has an opening 80a whose internal diameter is almost equal to the external diameter of the insertion part 16, and is attached to the front face of the front plate 54 and the back face of the rear plate 56, respectively. According to the wiper 80, not only drawing-in of foreign body between the mounting part 50 and the rotary body 44 can be prevented, but also entering of foreign body into the mounting part 50 can be prevented. In addition, in an embodiment after FIG. 7, the same members as those of the aforementioned embodiment will be designated by the same reference numerals, and the description thereof will be omitted.

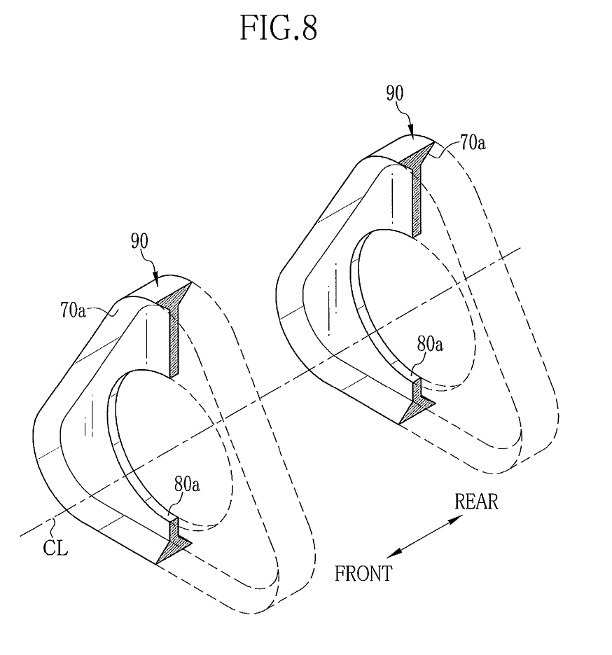

Moreover, in the above embodiment, the wiper of a tapered shape whose thickness becomes smaller toward its tip is used. In addition to this, however, as shown in FIG. 8, a wiper 90 having an outer periphery of triangular shape and a cross-section of a substantially T-shape may be used.

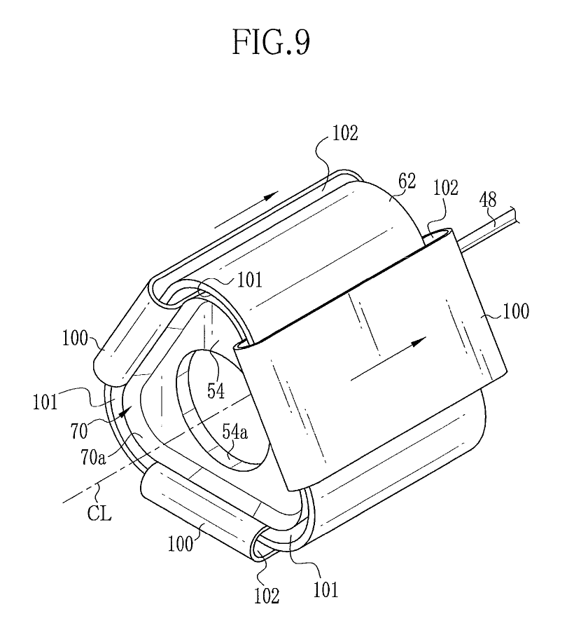

Additionally, the example in which the hollow toroidal rotary body is used has been described in the above embodiment. In addition to this, however, as shown in FIG. 9, an endless belt 100 may be used as the rotary body. In the example of FIG. 9, three endless belts 100 are arranged at intervals of 120° at the rotary body supporting tube 52 around the central axis CL. Since the components and configurations except for the endless belts 100 are same as the above embodiment, the same members as those of the aforementioned embodiment will be designated by the same reference numerals, and the description thereof will be omitted.

However, in the example of FIG. 9, since gaps 101 are formed between the front plate 54 (or the rear plate 56) and the wheel supporting tube 62, foreign body may enter from these gaps 101. In view of this, it is preferable to provide sealing members to close the gaps 101. The sealing members may be provided integrally with the wiper 70, or may be provided separately from the wiper 70 to be mounted on the wiper 70 or the wheel supporting tube 62.

On the wheel supporting tube 62, both sides of the endless belts 100 are open, and foreign body may enter from these openings 102. In view of this, it is preferable to provide side sealing members to close the openings 102. The side sealing members may be provided integrally with the wiper 70, or may be provided separately from the wiper 70 to be mounted on the wiper 70 or the wheel supporting tube 62.

It is also possible to provide a fixed barrel inside the gear barrel 58, and fix this fixed barrel to the insertion section 16. In this case, the gear barrel 58 is rotatably supported by the fixed barrel. Moreover, the front plate 54, the rear plate 56, and the wipers 70 are fixed on the outer periphery of the fixed barrel.

In addition, in the above embodiment, the example in which the invention is applied to an insertion assisting device of an electronic endoscope for medical diagnosis has been described. However, the invention can be applied to insertion assisting devices of conduit observation instruments referred to as other endoscopes and ultrasonic probes for industrial use or the like.

Various changes and modifications are possible in the present invention and may be understood to be within the present invention.

What is claimed is:

1. A self-propelled device for an endoscope comprising:
a mounting part detachably mounted on an insertion part of an endoscope;
a rotary body formed in a hollow toroidal shape or obtained by forming a belt in a ring shape;
a supporting part having at least a portion arranged in an internal space of the rotary body and supporting the rotary body so as to be able to circulate along the longitudinal direction of the insertion part, the supporting part being formed in the shape of a tube that surrounds the mounting part and attached to the outside of the mounting part with the rotary body supported;
a driving roller provided at the mounting part so as to come into contact with the rotary body, the driving roller circulating and moving the rotary body; and
first and second wipers respectively arranged at the front and rear ends of the mounting part so as to close a gap between the mounting part and the rotary body, the first and second wipers sliding on the rotary body when the rotary body rotates, thereby preventing drawing-in of foreign body between the mounting part and the rotary body.

2. The self-propelled device for an endoscope according to claim 1,
wherein the first and second wipers are formed from a material having elasticity, and are arranged in a state where the wipers are pressed against and elastically deformed by the rotary body.

3. The self-propelled device for an endoscope according to claim 2,
wherein the first and second wipers are arranged inside a turning point of the rotary body in a direction which said rotary body circulates and moves.

4. The self-propelled device for an endoscope according to claim 3,
wherein the first wiper is arranged with its surface being turned to the front end of the mounting part, and the angle between the surface and the rotary body is formed to be equal to or more than 90°, and
wherein the second wiper is arranged with its surface being turned to the rear end of the mounting part, and the angle between the surface and the rotary body is formed to be equal to or more than 90°.

5. The self-propelled device for an endoscope according to claim 3,
wherein the first and second wipers are formed in a tapered shape whose thickness becomes smaller toward its tip where the wiper slides on the rotary body.

6. The self-propelled device for an endoscope according to claim 3,
wherein the first and second wipers are formed in a ring shape, and are attached to the mounting part at a position where the tip of each of said first and second wipers extends outside in a radial direction of said mounting part.

7. The self-propelled device for an endoscope according to claim 3,
wherein the first and second wipers have cross-sections along the central axis of the insertion part formed in a substantially L-shape.

8. The self-propelled device for an endoscope according to claim 1,
Wherein the first and second wipers have cross-sections along the central axis of the insertion part formed in a substantially T-shape, and have a triangular head where its broad surface comes in contact with said rotary body.

9. The self-propelled device for an endoscope according to claim 3,
wherein the first and second wipers are formed from a biocompatible plastic.

10. The self-propelled device for an endoscope according to claim 3,
wherein the first and second wipers are detachably provided at the mounting part so as to be replaceable when the wipers have deteriorated through use.

11. The self-propelled device for an endoscope according to claim 3,
wherein the mounting part has an opening portion through which the insertion part is inserted, and is mounted on the outer periphery of the insertion part as the insertion part is inserted through the opening portion.

12. The self-propelled device for an endoscope according to claim 3,
wherein the rotary bodies are endless belts obtained by forming a belt in a ring shape, and are juxtaposed around the longitudinal direction of the insertion part.

13. The self-propelled device for an endoscope according to claim 12, further comprising:
a front end sealing part preventing entering of foreign body from a front gap formed between the supporting part and the mounting part between a pair of the adjacent endless belts; and
a rear end sealing part preventing entering of foreign body from a rear gap formed between the supporting part and the mounting part between the pair of the adjacent endless belts.

14. The self-propelled device for an endoscope according to claim 13, further comprising:
a side sealing part preventing entering of foreign body from side gaps formed at both sides of the endless belt.
